(19) United States
(12) Patent Application Publication
(10) Pub. No.: US 2019/0133137 A1
(43) Pub. Date: May 9, 2019

(54) COMPOSITIONS AND METHODS FOR INHIBITING SEED GERMINATION

(71) Applicant: Georgia State University Research Foundation, Inc., Atlanta, GA (US)

(72) Inventors: George E. Pierce, Canton, GA (US); Sidney A. Crow, Smyrna, GA (US)

(21) Appl. No.: 16/307,333

(22) PCT Filed: Jun. 6, 2017
(86) PCT No.: PCT/US2017/036130

§ 371 Date: Dec. 5, 2018

Related U.S. Application Data
(60) Provisional application No. 62/346,339, filed on Jun. 6, 2016.

(51) Int. Cl. A01N 63/02; C12N 1/20; C12N 9/88; C12N 9/82; C12N 9/02

(52) U.S. Cl. CPC A01N 63/02; C12N 1/20; C12N 9/88; C12N 9/82; C12N 9/0069; C12N 9/0083; C12Y 114/12011; C12Y 305/01001; C12Y 305/99007; C12Y 113/11; C12Y 114/15003; C12Y 114/99039; C12Y 402/01084

(57) ABSTRACT

Provided are methods and compositions for inhibiting seed germination. The methods comprise exposing a seed to a composition comprising one or more enzymes, one or more bacteria, and/or an enzymatic extract, wherein the one or more enzymes, one or more bacteria, and/or the enzymatic extract isolated from one or more bacteria are exposed to seed in a quantity sufficient to inhibit seed germination.

### CROSS-REFERENCE TO RELATED APPLICATIONS

This application claims the benefit of and priority to co-pending U.S. Provisional Patent Application No. 62/346,339, filed on Jun. 6, 2016, entitled “COMPOSITIONS AND METHODS FOR INHIBITING SEE GERMINATION”, the contents of which is incorporated by reference herein in its entirety.

### BACKGROUND

For items such as seed, from grains/cereals, where the seed may be stored for some time before use, it is common practice to “dry” the seed to a point which does not damage the seed but which makes the out-growth on contaminating microorganisms difficult. In general, the fungi (as compared to bacteria) have a higher tolerance to drying, and as a result, control of fungi is harder to control. Transient events which result in increased moisture (even if for a short time) can trigger the out-growth of fungi from the seed. Once the fungal growth becomes macroscopic, the fungus can rapidly spread resulting in irreversible, catastrophic damage to the seed. When seeds germinate, conditions can become permissive for fungal outgrowth. As such, there exists a need to inhibit or reduce seed germination to prevent undesirable outcomes, such as fungal outgrowth.

### SUMMARY

Provided herein are methods and compositions for inhibiting seed germination without the need for drying.

The methods comprise exposing an isolated seed to one or more bacteria, one or more enzymes, an enzymatic extract isolated from one or more bacteria, or any combination thereof, in a quantity sufficient to inhibit seed germination. The one or more bacteria can be selected from the group consisting of genus Rhodococcus, genus Brevibacterium, genus Pseudonocardia, genus Nocardia, genus Pseudomonas, and combinations thereof. The one or more enzymes can be selected from the group consisting of nitrile hydratases, amidases, asparaginases, ACC deaminases, cyanoalanine synthase-like enzymes, monooxygenases, dioxygenases, cyandiases, and combinations thereof.

The methods can involve exposing the seed to the one or more bacteria, one or more enzymes, or an enzymatic extract isolated from one or more bacteria, individually or in combination before drying, instead of drying, or in addition to drying. In some cases, exposed seeds are still allowed to dry, but to a lesser extent, which can improve viability of the seeds.

In some embodiments, where large quantities of seed are stored (as in a silo or grain elevator) the product could be formulated in such a way as to demonstrate magnetic properties (e.g. immobilized with a formulation incorporating magnetite, thus making the immobilized particle “magnetic”). Such magnetic materials could be easily separated from the grain. Or alternatively, the product could be incorporated into a filter material resulting in the treating of the air in the elevator/silo. In another embodiment, the product could be incorporated into a semi-permeable material, resulting in the free-exchange of gases, some liquid (if desired) but completing retaining the product inside and thus not in contact with the grain.

Such embodiments could be configured for applications involving covered hoppers (suitable for example for train, truck or boat/barge transportation) for small quantities of seed the product could be incorporated into the packaging containing the seed. (a washing step could be included with the product or alternatively based upon the length of time of seed germination inhibition, the product could be configured such that expose to the seed was conducted prior to placing the seed in packets, and thus relying on the residual effect of the product.)

In certain aspects, the one or more enzymes, enzymatic extract[s], and one or more bacteria, individually or in combination can be associated with, placed in, placed on, or embedded within an inanimate object or material. In certain aspects, the inanimate object or material is selected from the group consisting of a counter top, cardboard box, an inorganic surface, paper wrapping, wallboard, wood, medical device, and surgical dressing.

The details of one or more aspects are set forth in the accompanying drawings and description below. Other features, objects, and advantages will be apparent from the description and drawings and from the claims.

### DETAILED DESCRIPTION

As used herein, the singular forms “a”, “an”, “the”, include plural referents unless the context clearly dictates otherwise.

Throughout the specification the word “comprising,” or grammatical variations thereof, will be understood to imply the inclusion of a stated element, integer or step, or group of elements, integers or steps, but not the exclusion of any other element, integer or step, or group of elements, integers or steps.

The disclosed compositions, apparatuses, and methods arise from the finding that one or more bacteria are capable of inhibiting seed germination. Compositions, apparatuses, and methods as described may also inhibit or reduce fungal growth in addition to inhibiting or reducing seed germination. Optionally, the bacteria are induced to produce one or more enzymes capable of inhibiting seed germination. Optionally, as described herein the specific enzymatic activity of one or more enzymes are capable of inhibiting seed germination.

Provided herein are methods and compositions for inhibiting seed germination. The methods comprise exposing a seed to a composition comprising one or more bacteria, wherein the one or more bacteria are selected from the group consisting of genus Rhodococcus, genus Brevibacterium, genus Pseudonocardia, genus Nocardia, genus Pseudomonas and combinations thereof, and wherein the one or more bacteria are provided in a quantity sufficient to inhibit seed germination. Optionally, the bacteria are induced to produce one or more enzymes. In some embodiments, the methods comprise exposing a seed to a composition comprising one or more enzymes selected from the group consisting of nitrile hydratases, amidases, asparaginases, ACC deaminases, cyanoalanine synthase-like enzymes, monooxygenases, dioxygenases, cyanidases, and combinations thereof, wherein the enzymes are provided in a quantity sufficient to inhibit seed germination.

As used herein, “seed” may refer to one seed, one or more seeds, an isolated seed, or one or more isolated seeds.

In certain embodiments, the seed is from a fruit and/or a vegetable. A “fruit” or “vegetable” can include, but is not limited to, apples, apricots, asparagus, avocados, bananas, beans, cabbage, cantaloupe, cucumbers, eggplant, grapefruit, grapes, honeydew melons, lemons, lettuce, lima beans, limes, mangos, nectarines, okra, broccoli, oranges, papayas, peaches, peppers, pineapples, potatoes, pumpkins, soybeans, spinach, summer squash, sweet potatoes, tomatoes, watermelons, winter squash, and zucchini.

In certain embodiments, the seed is from a flower. A “flower” can include, but is not limited to, carnation, rose, orchid, portulca, malva, begonia, anthurium, cattleyas, and poinsettias.

In certain embodiments, the seed is a grain. Grains are seeds (with or without hull or fruit layers attached) harvested for human food or animal feed. Optionally, the grain is a cereal grain, a starchy grain, a grain legume or an oilseed. Cereal grains include, but are not limited to, maize or corn, sorghum, fonio, millet, e.g., pearl millet, proso millet, finger millet, foxtail millet, Japanese millet, kodo millet, Job's tears, rice, rye, barley, oat, triticale, wild rice, and teff. Starchy grains include, but are not limited to, amaranth, quinoa and buckwheat. Grain legumes includes but are not limited to soybean, common bean, chickpea, lima bean, runner bean, pigeon pea, lentil, field pea or garden pea, lupin, mung bean, fava bean, and peanut. Oilseeds includes but are not limited to, rapeseed (including canola), India mustard, black mustard, sunflower seed, safflower, flax seed (Flax family), hemp seed (Hemp family), and poppyseed (Poppy family). Optionally, the compositions comprising one or more bacteria or one or more enzymes are exposed to the grain in the field prior to or during harvesting of the grain. Optionally, the compositions are applied, e.g., coated, to grain or other seeds prior to planting.

In certain embodiments, the methods and compositions for inhibiting seed germination comprises exposing the seed to one or more bacteria selected from the group consisting of genus Rhodococcus, genus Brevibacterium, genus Pseudomonas, genus Nocardia, genus Pseudonocardia and combinations thereof. The one or more bacteria can, for example, include Rhodococcus spp. The Rhodococcus spp can, for example, include Rhodococcus rhodochrous DAP 96253 strain, Rhodococcus rhodochrous DAP 96622 strain, Rhodococcus erythropolis, or combinations thereof. Optionally, the compositions comprise Rhodococcus rhodochrous and Rhodococcus erythropolis. Exemplary organisms include, but are not limited to, Pseudomonas chloroaphis (ATCC 43051) (Gram-negative), Pseudomonas chloroaphis (ATCC 13985) (Gram-negative), Rhodococcus erythropolis (ATCC 47072) (Gram-positive), and Brevibacterium ketoglutamicum (ATCC 21533) (Gram-positive). Examples of Nocardia and Pseudonocardia species have been described in European Patent No. 0790310; Collins and Knowles J. Gen. Microbiol. 129:711-718 (1983); Harper Biochem. J. 165:309-319 (1977); Harper Int. J. Biochem. 17:677-683 (1985); Linton and Knowles J Gen. Microbiol. 132:1493-1501 (1986); and Yamaki et al., J. Ferm. Bioeng. 83:474-477 (1997).

Although in some embodiments the one or more bacteria are selected from the group consisting of Rhodococcus spp., Brevibacterium ketoglutamicum, and Pseudomonas chloroaphis, any bacterium that inhibits seed germination when exposed to seed can be used in the present methods. For example, bacteria belonging to the genus Nocardia [see Japanese Patent Application No. 54-129190], Rhodococcus [see Japanese Patent Application No. 2-470], Rhizobium [see Japanese Patent Application No. 5-236977], Klebsiella [Japanese Patent Application No. 5-30982], Aeromonas [Japanese Patent Application No. 5-30983], Agrobacterium [Japanese Patent Application No. 8-154691], Bacillus [Japanese Patent Application No. 8-187092], Pseudonocardia [Japanese Patent Application No. 8-56684], Burkholderia, Corynebacterium, and Pseudomonas are non-limiting examples of bacteria that can be used. Not all species within a given genus exhibit the same type of enzyme activity and/or production. Thus, it is possible to have a genus generally known to include strains capable of exhibiting a desired activity but have one or more strains that do not naturally exhibit the desired activity or one or more strains which do not exhibit the activity when grown on the same medium as the species which exhibit this activity. Thus, host microorganisms can include strains of bacteria that are not specifically known to have the desired activity but are from a genus known to have specific strains capable of producing the desired activity. Such strains can have transferred thereto one or more genes useful to cause the desired activity. Non-limiting examples of such strains include Rhodococcus equi and Rhododoccus globerulus PWD1.

Further, specific examples of bacteria include, but are not limited to, Nocardia sp., Rhodococcus sp., Rhodococcus rhodochrous, Klebsiella sp., Aeromonas sp., Citrobacter freundii, Agrobacterium rhizogenes, Agrobacterium tumefaciens, Xanthobacter flavas, Erwinia nigrifluens, Enterobacter sp., Streptomyces sp., Rhizobium sp., Rhizobium loti, Rhizobium legminosarum, Rhizobium merioti, Pantoea agglomerans, Klebsiella pneumoniae subsp. pneumoniae, Agrobacterium radiobacter, Bacillus smithii, Pseudonocardia thermophila, Pseudomonas chloroaphis, Rhodococcus erythropolis, Brevibacterium ketoglutamicum, and Pseudonocardia thermophila. Optionally, the microorganisms used can, for example, comprise Rhodococcus rhodochrous DAP 96253 and Rhodococcus rhodochrous DAP 96622, and combinations thereof.

As used herein, exposing the seed to one or more bacteria includes, for example, exposure to intact bacterial cells, bacterial cell lysates, and bacterial extracts that possess enzymatic activity (i.e., “enzymatic extracts”). Methods for preparing lysates and enzymatic extracts from cells, including bacterial cells, are routine in the art. Optionally, the one or more bacteria or enzymatic extracts are fixed with glutaraldehyde and crosslinked. Optionally, the crosslinked, glutaraldehyde-fixed bacteria or extract is formulated with a carrier into a spray.

In certain embodiments, the methods and compositions for inhibiting seed germination comprise exposing the seed to an enzyme. The enzyme can be selected from the group consisting of nitrile hydratase, amidase, asparaginase, ACC (1-aminocyclopropane-l-carboxylic acid) deaminase, cyanoalanine synthase-like enzyme, alkane monooxygenase, ammonium monooxygenase, methane monooxygenase, toluene dioxygenase, cyanidase, and/or a combination thereof. The enzyme can be provided within a composition for exposure to the seed. The enzyme can also be a purified enzyme or can be provided as an enzymatic extract as described above. Optionally, the methods for inhibiting seed germination comprise exposing the seed to a composition comprising an enzyme, the enzyme being selected from one or more of nitrile hydratase, amidase, asparaginase, ACC deaminase, cyanoalanine synthase-like enzyme, alkane monooxygenase, ammonium monooxygenase, methane monooxygenase, toluene dioxygenase, and cyanidase. The one or more bacteria, enzymatic extract[s], or one or more enzymes, individually or in combination, used in the methods may at times be more generally referred to herein as the “catalyst.”

In the methods provided herein, the seed is exposed to one or more bacteria, one or more enzymes, enzymatic extract isolated from or derived from the one or more bacteria, or any combination thereof, in a quantity sufficient to inhibit or reduce fungal growth. In some embodiments, the plant or plant part is exposed to one or more bacteria in combination with one or more exogenous enzymes and/or enzymatic extracts. “Exogenous” refers to enzymes or enzymatic extracts that are isolated and/or purified ex situ and is distinguished from enzymes produced by bacteria in situ. This combined exposure can take place simultaneously and/or sequentially. For example, the plant can be exposed to exogenous enzymes and/or enzymatic extracts 1 to 60 minutes, 1 to 24 hours, or 1 to 7 days after exposure to the bacteria. For example, the plant can be exposed to the exogenous enzymes and/or enzymatic extracts 1 to 10 minutes, 5 to 15 minutes, 10 to 20 minutes, 15 to 25 minutes, 20 to 30 minutes, 25 to 35 minutes, 30 to 40 minutes, 35 to 45 minutes, 40 to 50 minutes, 45 to 55 minutes, 50 to 60 minutes, 10 to 60 minutes, 20 to 50 minutes, or 30 to 40 minutes after exposure to the bacteria. For example, the plant can be exposed to the exogenous enzymes and/or enzymatic extracts 2 to 23 hours, 3 to 22 hours, 4 to 21 hours, 5 to 20 hours, 6 to 19 hours, 7 to 18 hours, 8 to 17 hours, 9 to 16 hours, 10 to 15 hours, 11 to 14 hours, or 12 to 13 hours after exposure to the bacteria. For example, the plant can be exposed to the exogenous enzymes and/or enzymatic extracts 2 to 6 days, 3 to 5 days, or 4 days after exposure to the bacteria.

In the methods provided herein, the seed is exposed to one or more bacteria, one or more enzymes, enzymatic extract isolated from or derived from the one or more bacteria, or any combination thereof, in a quantity sufficient to inhibit seed germination. In some embodiments, the plant or plant part is exposed to one or more bacteria in combination with one or more exogenous enzymes and/or enzymatic extracts. “Exogenous” refers to enzymes or enzymatic extracts that are isolated and/or purified ex situ and is distinguished from enzymes produced by bacteria in situ. This combined exposure can take place simultaneously and/or sequentially. For example, the plant can be exposed to exogenous enzymes and/or enzymatic extracts 1 to 60 minutes, 1 to 24 hours, or 1 to 7 days after exposure to the bacteria. For example, the plant can be exposed to the exogenous enzymes and/or enzymatic extracts 1 to 10 minutes, 5 to 15 minutes, 10 to 20 minutes, 15 to 25 minutes, 20 to 30 minutes, 25 to 35 minutes, 30 to 40 minutes, 35 to 45 minutes, 40 to 50 minutes, 45 to 55 minutes, 50 to 60 minutes, 10 to 60 minutes, 20 to 50 minutes, or 30 to 40 minutes after exposure to the bacteria. For example, the plant can be exposed to the exogenous enzymes and/or enzymatic extracts 2 to 23 hours, 3 to 22 hours, 4 to 21 hours, 5 to 20 hours, 6 to 19 hours, 7 to 18 hours, 8 to 17 hours, 9 to 16 hours, 10 to 15 hours, 11 to 14 hours, or 12 to 13 hours after exposure to the bacteria. For example, the plant can be exposed to the exogenous enzymes and/or enzymatic extracts 2 to 6 days, 3 to 5 days, or 4 days after exposure to the bacteria.

In the methods provided herein, the seed is exposed to one or more bacteria, one or more enzymes, enzymatic extract isolated from or derived from the one or more bacteria, or any combination thereof, in a quantity sufficient to inhibit fungal growth and seed germination. In some embodiments, the plant or plant part is exposed to one or more bacteria in combination with one or more exogenous enzymes and/or enzymatic extracts. “Exogenous” refers to enzymes or enzymatic extracts that are isolated and/or purified ex situ and is distinguished from enzymes produced by bacteria in situ. This combined exposure can take place simultaneously and/or sequentially. For example, the plant can be exposed to exogenous enzymes and/or enzymatic extracts 1 to 60 minutes, 1 to 24 hours, or 1 to 7 days after exposure to the bacteria. For example, the plant can be exposed to the exogenous enzymes and/or enzymatic extracts 1 to 10 minutes, 5 to 15 minutes, 10 to 20 minutes, 15 to 25 minutes, 20 to 30 minutes, 25 to 35 minutes, 30 to 40 minutes, 35 to 45 minutes, 40 to 50 minutes, 45 to 55 minutes, 50 to 60 minutes, 10 to 60 minutes, 20 to 50 minutes, or 30 to 40 minutes after exposure to the bacteria. For example, the plant can be exposed to the exogenous enzymes and/or enzymatic extracts 2 to 23 hours, 3 to 22 hours, 4 to 21 hours, 5 to 20 hours, 6 to 19 hours, 7 to 18 hours, 8 to 17 hours, 9 to 16 hours, 10 to 15 hours, 11 to 14 hours, or 12 to 13 hours after exposure to the bacteria. For example, the plant can be exposed to the exogenous enzymes and/or enzymatic extracts 2 to 6 days, 3 to 5 days, or 4 days after exposure to the bacteria.

“Exposing” a seed to one or more bacteria, one or more enzymes, and/or an enzymatic extract includes any method of presenting a bacterium, enzyme, and/or extract to the seed. Optionally, the seed is indirectly exposed to the one or more bacteria, one or more enzymes, and/or the enzymatic extract. Indirect methods of exposure include, for example, placing the one or more bacteria, one or more enzymes, and/or enzymatic extract in the general proximity of the seed (i.e., indirect exposure). Optionally, the seed is directly exposed to one or more bacteria, one or more enzymes, and/or the enzymatic extract, whereby the one or more bacteria, one or more enzymes, and/or enzymatic extract are in direct contact with the seed.

In certain embodiments, exposure of the bacteria, enzyme, and/or the enzymatic extract isolated from the bacteria can occur, for example, by providing the bacteria, enzyme, and/or enzymatic extract in liquid form and spraying it onto or near the seed. The bacteria, enzyme, and/or enzymatic extract can, for example, further comprise a liquid carrier. Liquid carriers can be selected from the group consisting of an aromatic hydrocarbon, a substituted naphthalene, a phthalic acid ester, an aliphatic hydrocarbon, an alcohol, and a glycol. Optionally, the liquid carrier can be a wax or similar type material coating, which could be applied to the plant as a liquid, but would be solid at ambient or lower temperatures. Optionally, the bacteria, enzyme and/or enzymatic extract are provided onto or near the seed by a fog or spray. For example, the bacteria, enzyme or enzymatic extract can be provided to the soil in the area where the fungi is to be controlled.

In certain embodiments, exposure of the one or more bacteria, one or more enzymes, and/or the enzymatic extract isolated from the bacteria can occur, for example, by providing the bacteria, enzyme, and/or enzymatic extract in solid form and dusting it onto or near the seed. The bacteria, enzyme, and/or enzymatic extract can, for example, further comprise a solid carrier. The solid carrier can be selected from the group consisting of a dust, a wettable powder, a water dispersible granule, and mineral fillers. Optionally, the solid carrier is a mineral filler. Mineral fillers can, for example, be selected from the group consisting of a calcite, a silica, a talc, a kaolin, a montmorillonite, and an attapulgite. Other solid supports for use with the bacteria, enzyme, and/or enzymatic extract are described herein.

In certain embodiments, exposure of the one or more bacteria, one or more enzymes, and/the enzymatic extract isolated from the bacteria can occur, for example, by providing the bacteria, enzyme, and/enzymatic extract as a composition including iron or another magnetic material. Iron-based compositions including ferrous metal matrices possess a magnetic attraction and can be used to deliver the bacteria, enzymes and/enzymatic extracts to materials, e.g., crops that are filtered or cleaned using a magnet. This process advantageously removes any unwanted metal pieces from the grain in addition to removing the provided compositions comprising the bacteria, enzymes or enzymatic extracts. By way of example, the one or more bacteria, one or more enzymes, or the enzymatic extract isolated from the bacteria can be applied to the grain, e.g., the grain crop, in the form of a spray. The grain crop is then harvested and the harvested grain is then processed through a machine or apparatus comprising a magnet to filter or clean the harvested grain and remove unwanted metal pieces as well as compositions comprising the bacteria, enzymes and/enzymatic extracts from the harvested grain.

In certain embodiments, the one or more bacteria, one or more enzymes, and/or enzymatic extract further comprise a coating, wherein the coating makes the one or more bacteria, one or more enzymes, and/or enzymatic extract water resistant. The coating can be selected from a hydrophobic fatty acid polyester coating or a wax. Optionally, the hydrophobic fatty acid polyester coating is a long chain fatty acid polyester derived from sucrose, sorbitol, sorbinose, glycerol, or raffinose.

Also provided herein are compositions for inhibiting seed germination. The compositions can, for example, comprise one or more bacteria, one or more enzymes, and/or one or more enzymatic extracts capable of inhibiting seed germination. The compositions can further comprise solid, liquid, and gelatinous carriers, as disclosed above, and/or media and media components for inducing and stabilizing the one or more bacteria, one or more enzymes, and/or enzymatic extracts, as disclosed below. Optionally, the compositions can be converted into pellet form for distribution or application to the plant or plant part. Compositions as described herein may also inhibit fungal growth.

As defined herein, a “sufficient” quantity or effective amount of the bacteria, enzyme, and/or enzymatic extract will depend on a variety of factors, including, but not limited to, the particular bacteria, enzyme, and/or enzymatic extract utilized in the method, the form in which the bacteria is exposed to the seed (e.g., as intact bacterial cells (dead or alive), cell lysates, enzymatic extracts, and/or enzymes as described above), the means by which the bacteria, enzyme, and/or enzymatic extract is exposed to the seed, the length of time of the exposure, and the type and amount of fungal signal compounds that result in the inhibition or reduction of fungal growth. Optionally, the quantity of bacteria exposed to the seed is in the range of 1 to 250 mg, 50-200 mg, 100-150 mg, or 100 mg of cell-dry weight or the equivalent thereof for enzymatic extracts and enzymes. For 1 mg of dry weight of cells, there can be 150-300 units of nitrile hydratase, 10-25 units of amidase, 7-15 units of cyanidase, 7-20 units of ACC deaminase, and 7-20 units of cyanoalanine synthase-like enzyme. By way of other examples, the quantity of bacteria exposed to the seed is in the range of 0.1 mg to 1 g, 0.1 to 400 mg, 1 to 200 mg, 1 to 80 mg, or 1 to 10 mg of cell-dry weight or the equivalent thereof for enzymatic extracts and enzymes. By way of example, the quantity of bacteria exposed to the seed is, for example, in the range of 0.1 mg to 1 g per 9-10 kilos (kg) of plant or plant part. It would be a matter of routine experimentation for the skilled artisan to determine the “sufficient” quantity of the one or more bacteria, one or more enzymes, or enzymatic extract necessary to inhibit seed germination. For example, if the bacteria, one or more enzymes, or enzymatic extract necessary to inhibit or reduce fungal growth are immobilized or stabilized, the quantity of bacteria, one or more enzymes, or enzymatic extract is adjusted to inhibit seed germination.

As defined herein, a “sufficient” quantity or effective amount of the bacteria, enzyme, and/or enzymatic extract will depend on a variety of factors, including, but not limited to, the particular bacteria, enzyme, and/or enzymatic extract utilized in the method, the form in which the bacteria is exposed to the seed (e.g., as intact bacterial cells (dead or alive), cell lysates, enzymatic extracts, and/or enzymes as described above), the means by which the bacteria, enzyme, and/or enzymatic extract is exposed to the seed, the length of time of the exposure, and the type and amount of compounds relating to seed germination that result in the inhibition or reduction of seed germination. Optionally, the quantity of bacteria exposed to the seed is in the range of 1 to 250 mg, 50-200 mg, 100-150 mg, or 100 mg of cell-dry weight or the equivalent thereof for enzymatic extracts and enzymes. For 1 mg of dry weight of cells, there can be 150-300 units of nitrile hydratase, 10-25 units of amidase, 7-15 units of cyanidase, 7-20 units of ACC deaminase, and 7-20 units of cyanoalanine synthase-like enzyme. By way of other examples, the quantity of bacteria exposed to the seed is in the range of 0.1 mg to 1 g, 0.1 to 400 mg, 1 to 200 mg, 1 to 80 mg, or 1 to 10 mg of cell-dry weight or the equivalent thereof for enzymatic extracts and enzymes. By way of example, the quantity of bacteria exposed to the seed is, for example, in the range of 0.1 mg to 1 g per 9-10 kilos (kg) of plant or plant part. It would be a matter of routine experimentation for the skilled artisan to determine the “sufficient” quantity of the one or more bacteria, one or more enzymes, or enzymatic extract necessary to inhibit or reduce seed germination. For example, if the bacteria, one or more enzymes, or enzymatic extract necessary to inhibit or reduce seed germination are immobilized or stabilized, the quantity of bacteria, one or more enzymes, or enzymatic extract is adjusted to inhibit seed germination.

As defined herein, a “sufficient” quantity or effective amount of the bacteria, enzyme, and/or enzymatic extract will depend on a variety of factors, including, but not limited to, the particular bacteria, enzyme, and/or enzymatic extract utilized in the method, the form in which the bacteria is exposed to the seed (e.g., as intact bacterial cells (dead or alive), cell lysates, enzymatic extracts, and/or enzymes as described above), the means by which the bacteria, enzyme, and/or enzymatic extract is exposed to the seed, the length of time of the exposure, and the type and amount of compounds relating to fungal growth and seed germination that result in the inhibition or reduction of seed germination and inhabitation or reduction of fungal growth.

Optionally, the quantity of bacteria exposed to the seed is in the range of 1 to 250 mg, 50-200 mg, 100-150 mg, or 100 mg of cell-dry weight or the equivalent thereof for enzymatic extracts and enzymes. For 1 mg of dry weight of cells, there can be 150-300 units of nitrile hydratase, 10-25 units of amidase, 7-15 units of cyanidase, 7-20 units of ACC deaminase, and 7-20 units of cyanoalanine synthase-like enzyme. By way of other examples, the quantity of bacteria exposed to the seed is in the range of 0.1 mg to 1 g, 0.1 to 400 mg, 1 to 200 mg, 1 to 80 mg, or 1 to 10 mg of cell-dry weight or the equivalent thereof for enzymatic extracts and enzymes. By way of example, the quantity of bacteria exposed to the seed is, for example, in the range of 0.1 mg to 1 g per 9-10 kilos (kg) of plant or plant part. It would be a matter of routine experimentation for the skilled artisan to determine the “sufficient” quantity of the one or more bacteria, one or more enzymes, or enzymatic extract necessary to inhibit or reduce seed germination and inhibit or reduce fungal growth. For example, if the bacteria, one or more enzymes, or enzymatic extract necessary to inhibit or reduce seed germination and fungal growth are immobilized or stabilized, the quantity of bacteria, one or more enzymes, or enzymatic extract is adjusted to inhibit seed germination and reduce or inhibit fungal growth.

In certain embodiments, the one or more bacteria are “induced” to exhibit a desired characteristic (e.g., the expression of a desired level of activity of an enzyme of the bacteria) by exposure or treatment with a suitable inducing agent. Inducing agents include, but are not limited to urea, methyl carbamate, cobalt, asparagine, glutamine, and combinations thereof. Optionally, the one or more bacteria are exposed to or treated with urea, methyl carbamate, methacrylamide, or acetamide. Optionally, the one or more bacteria are exposed to or treated with a mixture of inducing agents comprising urea or methyl carbamate and one or more of asparagine and cobalt. In some embodiments, the compositions and methods optionally exclude an inducing agent, such as cobalt.

The inducing agent, when used, can be added at any time during cultivation of the desired cells. For example, with respect to bacteria, the culture medium can be supplemented with an inducing agent prior to beginning cultivation of the bacteria. Alternately, the bacteria could be cultivated on a medium for a predetermined amount of time to grow the bacteria and the inducing agent could be added at one or more predetermined times to induce the desired enzymatic activity in the bacteria. Moreover, the inducing agent could be added to the growth medium (or to a separate mixture including the previously grown bacteria) to induce the desired activity in the bacteria after the growth of the bacteria is completed or during a second growth or maintenance phase.

While not intending to be limited to a particular mechanism, “inducing” the bacteria may result in the production or activation (or increased production or increased activity) of one or more of enzymes, such as nitrile hydratase, amidase, asparaginase, ACC deaminase, cyanoalanine synthase-like enzyme, alkane monooxygenase, ammonium monooxygenase, methane monooxygenase, toluene dioxygenase, and/or cyanidase, and the induction of one or more of these enzymes may play a role in inhibiting or reducing fungal growth. “Nitrile hydratases,” “amidases,” “asparaginases,” “ACC deaminases,” “cyanoalanine synthase-like enzymes,” “AMO-type (alkane or ammonium) monooxygenases,” “methane monooxygenases,” “toluene dioxygenases,” and “cyanidases” comprise families of enzymes present in cells from various organisms, including but not limited to, bacteria, fungi, plants, and animals. Such enzymes are well known, and each class of enzyme possesses recognized enzymatic activities.

While not intending to be limited to a particular mechanism, “inducing” the bacteria may result in the production or activation (or increased production or increased activity) of one or more of enzymes, such as nitrile hydratase, amidase, asparaginase, ACC deaminase, cyanoalanine synthase-like enzyme, alkane monooxygenase, ammonium monooxygenase, methane monooxygenase, toluene dioxygenase, and/or cyanidase, and the induction of one or more of these enzymes may play a role in inhibiting or reducing seed germination. “Nitrile hydratases,” “amidases,” “asparaginases,” “ACC deaminases,” “cyanoalanine synthase-like enzymes,” “AMO-type (alkane or ammonium) monooxygenases,” “methane monooxygenases,” “toluene dioxygenases,” and “cyanidases” comprise families of enzymes present in cells from various organisms, including but not limited to, bacteria, fungi, plants, and animals. Such enzymes are well known, and each class of enzyme possesses recognized enzymatic activities.

While not intending to be limited to a particular mechanism, “inducing” the bacteria may result in the production or activation (or increased production or increased activity) of one or more of enzymes, such as nitrile hydratase, amidase, asparaginase, ACC deaminase, cyanoalanine synthase-like enzyme, alkane monooxygenase, ammonium monooxygenase, methane monooxygenase, toluene dioxygenase, and/or cyanidase, and the induction of one or more of these enzymes may play a role in inhibiting or reducing seed germination and fungal growth. “Nitrile hydratases,” “amidases,” “asparaginases,” “ACC deaminases,” “cyanoalanine synthase-like enzymes,” “AMO-type (alkane or ammonium) monooxygenases,” “methane monooxygenases,” “toluene dioxygenases,” and “cyanidases” comprise families of enzymes present in cells from various organisms, including but not limited to, bacteria, fungi, plants, and animals. Such enzymes are well known, and each class of enzyme possesses recognized enzymatic activities.

The methods of inducing an enzymatic activity can be accomplished without the requirement of introducing hazardous nitriles, such as acrylonitrile, into the environment.

Previously, it was believed that induction of specific enzyme activity in certain microorganisms required the addition of chemical inducers. For example, in the induction of nitrile hydratase activity in Rhodococcus rhodochrous and Pseudomonas chloroaphis, it was generally believed to be necessary to supplement with hazardous chemicals, such as acetonitrile, acrylonitrile, acrylamide, and the like. However, enzymatic activity in nitrile hydratase producing microorganisms can be induced with the use of non-hazardous media additives, such as amide containing amino acids and derivates thereof, and optionally stabilized with trehalose. Optionally, asparagine, glutamine, or combinations thereof, can be used as inducers. Methods of inducing and stabilizing enzymatic activity in microorganisms are described in U.S. Pat. No. 7,531,343 and U.S. Pat. No. 7,531,344, which are incorporated herein by reference.

The disclosed methods of inducing enzymatic activity provide for the production and stability of a number of enzymes using modified media, immobilization, and stabilization techniques, as described herein. For example, enzymatic activity can be induced and stabilized through use of media comprising amide-containing amino acids, or derivatives thereof, and, optionally stabilized by, trehalose. In some embodiments, the methods of induction and stabilization comprise culturing a nitrile hydratase producing microorganism in a medium comprising one or more amide containing amino acids or derivatives thereof, and, optionally, trehalose. Optionally, disclosed are methods for inducing nitrile-hydratase using a medium supplemented with amide containing amino acids or derivatives thereof, which preferably include asparagine, glutamine or a combination thereof. Optionally, disclosed are methods for inducing nitrile-hydratase using a nutritionally complete medium supplemented with only asparagine. Optionally, disclosed are methods for inducing nitrile-hydratase using a nutritionally complete medium supplemented with only glutamine. Optionally, disclosed are methods for stabilizing nitrile-hydratase using a nutritionally complete medium supplemented with only trehalose. More particularly, the methods of induction and stabilization comprise culturing the microorganism in the medium and optionally collecting the cultured microorganisms or enzymes produced by the microorganisms.

Induction and stabilization of enzymes can be achieved without the use of hazardous nitriles. However, while the induction methods eliminate the need for hazardous chemicals for enzyme activity induction, the use of such further inducers is not excluded. For example, one or more nitriles could be used to assist in specific activity development. Media supplemented with succinonitrile and cobalt can be useful for induction of enzymes, including, for example, nitrile hydratase, amidase, asparaginase I, ACC deaminase, cyanoalanine synthase-like enzyme, alkane monooxygenase, ammonium monooxygenase, methane monooxygenase, toluene dioxygenase, and cyanidase. However, the use of nitriles is not necessary for induction of enzyme activity. While the use of nitriles and other hazardous chemicals is certainly not preferred, optionally, such use is possible.

Stabilization of enzyme activity can be achieved through immobilization methods, such as affixation, entrapment, and cross-linking, thereby, extending the time during which enzyme activity can be used. Thus, in some embodiments, induction methods and methods of delaying a chill injury response further comprise at least partially immobilizing the microorganism. Stabilization can be provided by immobilizing the enzymes, enzymatic extracts, or microorganisms producing the enzymes or enzymatic extracts. For example, enzymes or enzymatic extracts harvested from the microorganisms or the induced microorganisms themselves can be immobilized to a substrate as a means to stabilize the induced activity. Optionally, the nitrile hydratase producing microorganisms are at least partially immobilized. Optionally, the enzymes or microorganisms are at least partially entrapped in or located on the surface of a substrate. This allows for presentation of an immobilized material with induced activity (e.g., a catalyst) in such a manner as to facilitate reaction of the catalyst with an intended material and recovery of a desired product while simultaneously retaining the catalyst in the reaction medium and in a reactive mode. In certain embodiments, the stabilization through immobilization methods, such as affixation and entrapment, of the one or more bacteria kills or inactivates the one or more bacteria. Thus, optionally, the induced microorganisms used in the present methods are dead (killed) or inactivated, but are still capable of exhibiting catalyst activity.

Any substrate generally useful for affixation of enzymes, enzymatic extracts, or microorganisms can be used. Optionally, the substrate comprises alginate or salts thereof.

Alginate is a linear copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks), alternating M and G-residues (MG-blocks), or randomly organized blocks. Optionally, calcium alginate is used as the substrate. The calcium alginate can, for example, be cross-linked, such as with polyethyleneimine, to form a hardened calcium alginate substrate. Further description of such immobilization techniques can be found in Bucke, “Cell Immobilization in Calcium Alginate,” Methods in Enzymology, vol. 135, Part B (ed. K. Mosbach) pp. 175-189 (1987), which is incorporated herein by reference. The stabilization effect of immobilization using polyethylenimine cross-linked calcium alginate is discussed in U.S. patent application Ser. No. 11/695,377, filed Apr. 2, 2007, which is hereby incorporated by reference in its entirety.

Optionally, the substrate comprises an amide-containing polymer. Any polymer comprising one or more amide groups can be used. Optionally, the substrate comprises a polyacrylamide polymer.

Stabilization can further be achieved through cross-linking. For example induced microorganisms can be chemically cross-linked to form agglutinations of cells. Optionally, the induced microorganisms are fixed and cross-linked using glutaraldehyde. For example, microorganisms can be suspended in a mixture of de-ionized water and glutaraldehyde followed by addition of polyethylenimine until maximum flocculation is achieved. The cross-linked microorganisms (typically in the form of particles formed of a number of cells) can be harvested by simple filtration. Further description of such techniques is provided in Lopez-Gallego, et al., J. Biotechnol. 119:70-75 (2005), which is incorporated herein by reference. In certain embodiments, the cross-linking kills or inactivates the microorganism. Thus, optionally, the induced microorganisms used in the present methods are dead (killed) or inactivated, but are still capable of exhibiting catalyst activity.

Optionally, the microorganisms, enzymes, and/or enzymatic extracts can be encapsulated rather than allowed to remain in the classic Brownian motion. Such encapsulation facilitates collection, retention, and reuse of the microorganisms and generally comprises affixation of the microorganisms to a substrate. Such affixation can also facilitate stabilization of the microorganisms, enzymes, and/or enzymatic extracts as described above, or may be solely to facilitate ease of handling of the induced microorganisms, enzymes, or enzymatic extracts.

The microorganisms, enzymes, and/or enzymatic extracts can be immobilized by any method generally recognized for immobilization of microorganisms, enzymes, and/or enzymatic extracts such as sorption, electrostatic bonding, covalent bonding, and the like. Generally, the microorganisms, enzymes, and/or enzymatic extracts are immobilized or entrapped on a solid support which aids in the recovery of the microorganisms enzymes, or enzymatic extracts from a mixture or solution, such as a detoxification reaction mixture. Suitable solid supports include, but are not limited to, granular activated carbon, compost, wood or wood products, (e.g., paper, wood chips, wood nuggets, shredded pallets or trees), bran (e.g., wheat bran), metal or metal oxide particles (e.g., alumina, ruthenium, iron oxide), ion exchange resins, DEAE cellulose, DEAE-SEPHADEX® polymer, waxes/coating materials (such as those used as a coating for fruits and vegetables and inanimate surfaces), ceramic beads, cross-linked polyacrylamide beads, cubes, prills, or other gel forms, alginate beads, κ-carrageenan cubes, as well as solid particles that can be recovered from the aqueous solutions due to inherent magnetic ability. A solid support can also include physical structures, inanimate objects, and materials as described below. The shape of the catalyst is variable (in that the desired dynamic properties of the particular entity are integrated with volume/surface area relationships that influence catalyst activity). Optionally, the induced microorganism is immobilized in alginate beads that have been cross-linked with polyethyleneimine or is immobilized in a polyacrylamide-type polymer.

In some embodiments, the compositions and medium used in the induction and stabilization methods further comprise one or more amide containing amino acids or derivatives thereof, and/or trehalose. The amide containing amino acids can, for example, be selected from the group consisting of asparagine, glutamine, derivatives thereof, or combinations thereof. For example, the amide-containing amino acids may include natural forms of asparagine, anhydrous asparagine, asparagine monohydrate, or natural forms of glutamine, anhydrous glutamine, and/or glutamine monohydrate, each in the form of the L-isomer or D-isomer.

The concentration of the amide containing amino acids or derivatives thereof in the medium can vary depending upon the desired end result of the culture. For example, a culture may be carried out for the purpose of producing microorganisms having a specific enzymatic activity. Optionally, a culture may be carried out for the purpose of forming and collecting a specific enzyme from the cultured microorganisms. Optionally, a culture may be carried out for the purpose of forming and collecting a plurality of enzymes having the same or different activities and functions.

The amount of the amide containing amino acids, or derivatives thereof, added to the growth medium or mixture can generally be up to 10,000 parts per million (ppm) (i.e., 1% by weight) based on the overall weight of the medium or mixture. The induction methods are particularly beneficial, however, in that enzyme activity can be induced through addition of even lesser amounts. Optionally, the one or more amide containing amino acids are present at a concentration of at least 50 ppm. By way of other examples, the concentration of the amide containing amino acids or derivatives thereof is in the range of 50 ppm to 5,000 ppm, 100 ppm to 3,000 ppm, 200 ppm to 2,000 ppm, 250 ppm to 1500 ppm, 500 ppm to 1250 ppm, or 500 ppm to 1000 ppm.

In some embodiments, the stabilization methods include the use of trehalose. The concentration of trehalose in the compositions or medium used in the induction methods can be at least 1 gram per liter (g/L). Optionally, the concentration of trehalose is in the range of lg/L to 50 g/L, or 1 g/L to 10 g/L. Optionally, the concentration of trehalose in the medium is at least 4 g/L.

The amide containing amino acids or derivatives thereof and/or trehalose are added to a nutritionally complete media. A suitable nutritionally complete medium generally is a growth medium that can supply a microorganism with the necessary nutrients required for its growth, which minimally includes a carbon and/or nitrogen source. One specific example is the commercially available R2A agar medium, which typically consists of agar, yeast extract, proteose peptone, casein hydrolysate, glucose, soluble starch, sodium pyruvate, dipotassium hydrogenphosphate, and magnesium sulfate. Another example of a nutritionally complete liquid medium is Yeast Extract Malt Extract Agar (YEMEA), which consists of glucose, malt extract, and yeast extract (but specifically excludes agar). Also, media of similar composition, but of vegetable origin can be used for the disclosed methods. Any nutritionally complete medium known in the art could be used for the disclosed methods, the above media being described for exemplary purposes only. Such nutritionally complete media can be included in the compositions described herein.

Optionally, the disclosed compositions and media can contain further additives. Typically, the other supplements or nutrients are those useful for assisting in greater cell growth, greater cell mass, or accelerated growth. For example, the compositions and media can comprise a carbohydrate source in addition to any carbohydrate source already present in the nutritionally complete medium.

As described above, most media typically contain some content of carbohydrate (e.g., glucose); however, it can be useful to include an additional carbohydrate source (e.g., maltose or less refined sugars, such as dextrose equivalents that would be polymers of dextrose, or any carbohydrate that supports growth of the cell and induction of the desired activity). The type of excess carbohydrate provided can depend upon the desired outcome of the culture. For example, the addition of carbohydrates, such as maltose or maltodextrin, has been found to provide improved induction of asparaginase I. Additionally, the addition of carbohydrates, such as maltose or maltodextrin, potentially improves stability of enzymatic activity (e.g., nitrile hydratase activity).

In some embodiments, the compositions and media further comprise cobalt. Cobalt or a salt thereof can be added to the mixture or media. For example, the addition of cobalt (e.g., cobalt chloride) to the media can be particularly useful for increasing the mass of the enzyme produced by the cultured microorganisms. Cobalt or a salt thereof can, for example, be added to the culture medium such that the cobalt concentration is an amount up to 400 ppm. Cobalt can, for example, be present at a concentration of 5 ppm to 400 ppm, 10 ppm to 100 ppm, 10 ppm to 80 ppm, or 10 ppm to 25 ppm.

In some embodiments, the compositions and media further comprise urea. Urea or a salt thereof can be added to the mixture or media. Urea or a salt thereof can, for example, be added to the culture medium such that the urea concentration is in an amount up to 10 g/L. Urea can, for example, be present in a concentration of 5 g/L to 30 g/L, 5 g/L to 20 g/L, 5 g/L to 12 g/L, or 7 g/L to 10 g/L. Optionally, urea is present at a concentration of 7.5 g/L. Optionally, both urea and cobalt are added to the media.

The compositions and media may also include further components. For example, other suitable medium components may include commercial additives, such as cottonseed protein, maltose, maltodextrin, and other commercial carbohydrates. Optionally, the medium further comprises maltose or maltodextrin. Maltose or maltodextrin, for example, can be added to the culture medium such that the maltose or maltodextrin concentration is at least 1 g/L. Optionally, the compositions and media are free of any nitrile containing compounds. Nitrile compounds were previously required in the culture medium to induce enzyme activity toward two or more nitrile compounds. The compositions described herein achieve this through the use of completely safe trehalose and/or amide containing amino acids or derivatives thereof; therefore, the medium can be free of any nitrile containing compounds.

“Enzymatic activity,” as used herein, generally refers to the ability of an enzyme to act as a catalyst in a process, such as the conversion of one compound to another compound. Likewise, the desired activity referred to herein can include the activity of one or more enzymes being actively expressed by one or more microorganisms. In particular, nitrile hydratase catalyzes the hydrolysis of nitrile (or cyanohydrin) to the corresponding amide (or hydroxy acid). Amidase catalyzes the hydrolysis of an amide to the corresponding acid or hydroxy acid. Similarly, an asparaginase enzyme, such as asparaginase I, catalyzes the hydrolysis of asparagine to aspartic acid. ACC deaminase catalyzes the hydrolysis of 1-aminocyclopropane-1-carboxylate to ammonia and α-ketobutyrate. Beta-cyanoalanine synthase catalyzes the formation of the non-protein amino acid cyanoalanine from cysteine and cyanide. Cyanidase catalyzes the hydrolysis of cyanide to ammonia and formate. Alkane or ammonium monooxygenase (AMO) and methane monooxygenase catalyze the hydrolysis of ethylene to ethylene epoxide. Toluene dioxygenase can, for example, oxidize ethylene, and is known as an AMO-like enzyme. Ethylene degradation activity results in the degradation of produced ethylene.

Activity can be referred to in terms of “units” per mass of enzyme or cells (typically based on the dry weight of the cells, e.g., units/mg cdw). A “unit” generally refers to the ability to convert a specific content of a compound to a different compound under a defined set of conditions as a function of time. Optionally, one “unit” of nitrile hydratase activity refers to the ability to convert 1 μmol of acrylonitrile to its corresponding amide per minute, per milligram of cells (dry weight) at a pH of 7.0 and a temperature of 30° C. Similarly, one unit of amidase activity refers to the ability to convert 1 μmol of acrylamide to its corresponding acid per minute, per milligram of cells (dry weight) at a pH of 7.0 and a temperature of 30° C. Further, one unit of asparaginase I activity refers to the ability to convert 1 μmol of asparagine to its corresponding acid per minute, per milligram of cells (dry weight) at a pH of 7.0 and a temperature of 30° C. Further, one unit of ACC deaminase activity refers to the ability to convert 1 μmol of 1-aminocyclopropane-1-carboxylate to ammonia and α-ketobutyrate per minute, per milligram of cells (dry weight) at a pH of 7.0 and a temperature of 30° C. Further, one unit of cyanoalanine synthase-like enzyme activity refers to the ability to convert 1 μmol of cysteine and cyanide to cyanoalanine per minute, per milligram of cells (dry weight) at a pH of 7.0 and a temperature of 30° C. Further, one unit of cyanidase activity refers to the ability to convert 1 μmol of cyanide to ammonia and formate per minute, per milligram of cells (dry weight) at a pH of 7.0 and a temperature of 30° C. Further, one unit of alkane or ammonium monooxygenase (AMO) or methane monooxygenase activity refers to the ability to convert 1 μmol of ethylene to ethylene epoxide. Further, one unit of toluene dioxygenase refers to the ability to convert 1 μmol of ethylene to ethylene epoxide. Assays for measuring nitrile hydratase activity, amidase activity, asparaginase activity, ACC deaminase activity, cyanoalanine synthase-like enzyme activity, alkane or ammonium monooxygenase (AMO) activity, methane monooxygenase activity, toluene dioxygenase (AMO-like) activity, and cyanidase activity are known in the art and include, for example, the detection of free ammonia. See, e.g., Fawcett and Scott, J. Clin. Pathol. 13:156-9 (1960).

Generally, any bacterial, fungal, plant, or animal cell capable of producing or being induced to produce nitrile hydratase, amidase, asparaginase, ACC deaminase activity, cyanoalanine synthase-like enzyme activity, alkane or ammonium monooxygenase (AMO) activity, methane monooxygenase activity, toluene dioxygenase activity, and cyanidase activity, or any combination thereof may be used herein. A nitrile hydratase, amidase, asparaginase, ACC deaminase, cyanoalanine synthase-like enzyme, alkane or ammonium monooxygenase, methane monooxygenase, toluene dioxygenase, and/or cyanidase may be produced constitutively in a cell from a particular organism (e.g., a bacterium, fungus, plant cell, or animal cell) or, alternatively, a cell may produce the desired enzyme or enzymes only following “induction” with a suitable inducing agent. “Constitutively” is intended to mean that at least one enzyme disclosed herein is continually produced or expressed in a particular cell type. Other cell types, however, may need to be “induced,” as described above, to express nitrile hydratase, amidase, asparaginase, ACC deaminase, cyanoalanine synthase-like enzyme, alkane or ammonium monooxygenase, methane monooxygenase, toluene dioxygenase, and cyanidase at a sufficient quantity or enzymatic activity level to inhibit fungal growth. That is, an enzyme disclosed herein may only be produced (or produced at sufficient levels) following exposure to or treatment with a suitable inducing agent. Such inducing agents are known and outlined above. For example, the one or more bacteria are treated with an inducing agent such as urea, methyl carbamate, cobalt, asparagine, glutamine, or any mixture thereof, more particularly urea or methyl carbamate optionally in combination with asparagine or cobalt. Furthermore, as disclosed in U.S. Pat. Nos. 7,531,343 and 7,531,344, which are incorporated by reference in their entireties, entitled “Induction and Stabilization of Enzymatic Activity in Microorganisms,” asparaginase I activity can be induced in Rhodococcus rhodochrous DAP 96622 (Gram-positive) or Rhodococcus rhodochrous DAP 96253 (Gram-positive), in medium supplemented with amide containing amino acids or derivatives thereof. Other strains of Rhodococcus can also preferentially be induced to exhibit asparaginase I enzymatic activity utilizing amide containing amino acids or derivatives thereof.

Generally, any bacterial, fungal, plant, or animal cell capable of producing or being induced to produce nitrile hydratase, amidase, asparaginase, ACC deaminase activity, cyanoalanine synthase-like enzyme activity, alkane or ammonium monooxygenase (AMO) activity, methane monooxygenase activity, toluene dioxygenase activity, and cyanidase activity, or any combination thereof may be used herein. A nitrile hydratase, amidase, asparaginase, ACC deaminase, cyanoalanine synthase-like enzyme, alkane or ammonium monooxygenase, methane monooxygenase, toluene dioxygenase, and/or cyanidase may be produced constitutively in a cell from a particular organism (e.g., a bacterium, fungus, plant cell, or animal cell) or, alternatively, a cell may produce the desired enzyme or enzymes only following “induction” with a suitable inducing agent. “Constitutively” is intended to mean that at least one enzyme disclosed herein is continually produced or expressed in a particular cell type. Other cell types, however, may need to be “induced,” as described above, to express nitrile hydratase, amidase, asparaginase, ACC deaminase, cyanoalanine synthase-like enzyme, alkane or ammonium monooxygenase, methane monooxygenase, toluene dioxygenase, and cyanidase at a sufficient quantity or enzymatic activity level to inhibit seed germination. That is, an enzyme disclosed herein may only be produced (or produced at sufficient levels) following exposure to or treatment with a suitable inducing agent. Such inducing agents are known and outlined above. For example, the one or more bacteria are treated with an inducing agent such as urea, methyl carbamate, cobalt, asparagine, glutamine, or any mixture thereof, more particularly urea or methyl carbamate optionally in combination with asparagine or cobalt. Furthermore, as disclosed in U.S. Pat. Nos. 7,531,343 and 7,531,344, which are incorporated by reference in their entireties, entitled “Induction and Stabilization of Enzymatic Activity in Microorganisms,” asparaginase I activity can be induced in Rhodococcus rhodochrous DAP 96622 (Gram-positive) or Rhodococcus rhodochrous DAP 96253 (Gram-positive), in medium supplemented with amide containing amino acids or derivatives thereof. Other strains of Rhodococcus can also preferentially be induced to exhibit asparaginase I enzymatic activity utilizing amide containing amino acids or derivatives thereof.

P. chloroaphis (ATCC Deposit No. 43051), which produces asparaginase I activity in the presence of asparagine and ACC deaminase, and B. kletoglutamicum (ATCC Deposit No. 21533), a Gram-positive bacterium that has also been shown to produce asparaginase activity, are also used in the disclosed methods. Fungal cells, such as those from the genus Fusarium, plant cells, and animal cells, that express a nitrile hydratase, amidase, and/or an asparaginase, may also be used herein, either as whole cells or as a source from which to isolate one or more of the above enzymes.

The nucleotide and amino acid sequences for several nitrile hydratases, amidases, and asparaginases (e.g., type I asparaginases) from various organisms are disclosed in publicly available sequence databases. A non-limiting list of representative nitrile hydratases and aliphatic amidases known in the art is set forth in Tables 1 and 2 and in the sequence listing. The “protein score” referred to in Tables 1 and 2 provide an overview of percentage confidence intervals (% Confid. Interval) of the identification of the isolated proteins based on mass spectroscopy data.

TABLE 1
Amino Acid Sequence Information for Representative
Nitrile Hydratases

| | | Protein Score |
|---|---|---|
| Source organism | Accession No. | (% Confid. Interval) |
| Rhodococcus sp. | 806580 | 100% |
| Nocardia sp. | 27261874 | 100% |
| Rhodococcus rhodochrous | 49058 | 100% |
| Uncultured bacterium (BD2); | 27657379 | 100% |
| beta-subunit of | | |
| nitrile hydratase | | |
| Rhodococcus sp. | 806581 | 100% |
| Rhodococcus rhodochrous | 581528 | 100% |
| Uncultured bacterium (SP1); | 7657369 | 100% |
| alpha-subunit of | | |
| nitrile hydratase | | |

TABLE 2
Amino Acid Sequence Information for Representative
Aliphatic Amidases

| | | Protein Score |
|---|---|---|
| Source organism | Accession No. | (% Confid. Interval) |
| Rhodococcus rhodochrous | 62461692 | 100% |
| Nocardia farcinica IFM 10152 | 54022723 | 100% |
| Pseudomonas aeruginosa PAO1 | 15598562 | 98.3% |
| Helicobacter pylori J99 | 15611349 | 99.6% |
| Helicobacter pylori 26695 | 2313392 | 97.7% |
| Pseudomonas aeruginosa | 150980 | 94% |

Optionally, host cells that have been genetically engineered to express a nitrile hydratase, amidase, asparaginase, ACC deaminase, cyanoalanine synthase-like enzyme, alkane monooxygenase, toluene dioxygenase, and/or cyanidase can be exposed to a seed for inhibiting or reducing fungal growth or development of fungal growth. Specifically, a polynucleotide that encodes a nitrile hydratase, amidase, asparaginase, ACC deaminase, cyanoalanine synthase-like enzyme, alkane or ammonium monooxygenase, methane monooxygenase, toluene dioxygenase, or cyanidase (or multiple polynucleotides each of which encodes a nitrile hydratase, amidase, asparaginase, ACC deaminase, cyanoalanine synthase-like enzyme, alkane or ammonium monooxygenase, methane monooxygenase, toluene dioxygenase, or cyanidase) may be introduced by standard molecular biology techniques into a host cell to produce a transgenic cell that expresses one or more of the enzymes. The use of the terms “polynucleotide,” “polynucleotide construct,” “nucleotide,” or “nucleotide construct” is not intended to limit to polynucleotides or nucleotides comprising DNA. Those of ordinary skill in the art will recognize that polynucleotides and nucleotides can comprise ribonucleotides and combinations of ribonucleotides and deoxyribonucleotides. Such deoxyribonucleotides and ribonucleotides include both naturally occurring molecules and synthetic analogues. The polynucleotides described herein encompass all forms of sequences including, but not limited to, single-stranded forms, double-stranded forms, and the like.

Optionally, host cells that have been genetically engineered to express a nitrile hydratase, amidase, asparaginase, ACC deaminase, cyanoalanine synthase-like enzyme, alkane monooxygenase, toluene dioxygenase, and/or cyanidase can be exposed to a seed for inhibiting or reducing seed germination. Specifically, a polynucleotide that encodes a nitrile hydratase, amidase, asparaginase, ACC deaminase, cyanoalanine synthase-like enzyme, alkane or ammonium monooxygenase, methane monooxygenase, toluene dioxygenase, or cyanidase (or multiple polynucleotides each of which encodes a nitrile hydratase, amidase, asparaginase, ACC deaminase, cyanoalanine synthase-like enzyme, alkane or ammonium monooxygenase, methane monooxygenase, toluene dioxygenase, or cyanidase) may be introduced by standard molecular biology techniques into a host cell to produce a transgenic cell that expresses one or more of the enzymes. The use of the terms “polynucleotide,” “polynucleotide construct,” “nucleotide,” or “nucleotide construct” is not intended to limit to polynucleotides or nucleotides comprising

DNA. Those of ordinary skill in the art will recognize that polynucleotides and nucleotides can comprise ribonucleotides and combinations of ribonucleotides and deoxyribonucleotides. Such deoxyribonucleotides and ribonucleotides include both naturally occurring molecules and synthetic analogues. The polynucleotides described herein encompass all forms of sequences including, but not limited to, single-stranded forms, double-stranded forms, and the like.

Optionally, host cells that have been genetically engineered to express a nitrile hydratase, amidase, asparaginase, ACC deaminase, cyanoalanine synthase-like enzyme, alkane monooxygenase, toluene dioxygenase, and/or cyanidase can be exposed to a seed for inhibiting or reducing seed germination and inhibiting or reducing fungal growth. Specifically, a polynucleotide that encodes a nitrile hydratase, amidase, asparaginase, ACC deaminase, cyanoalanine synthase-like enzyme, alkane or ammonium monooxygenase, methane monooxygenase, toluene dioxygenase, or cyanidase (or multiple polynucleotides each of which encodes a nitrile hydratase, amidase, asparaginase, ACC deaminase, cyanoalanine synthase-like enzyme, alkane or ammonium monooxygenase, methane monooxygenase, toluene dioxygenase, or cyanidase) may be introduced by standard molecular biology techniques into a host cell to produce a transgenic cell that expresses one or more of the enzymes. The use of the terms “polynucleotide,” “polynucleotide construct,” “nucleotide,” or “nucleotide construct” is not intended to limit to polynucleotides or nucleotides comprising DNA. Those of ordinary skill in the art will recognize that polynucleotides and nucleotides can comprise ribonucleotides and combinations of ribonucleotides and deoxyribonucleotides. Such deoxyribonucleotides and ribonucleotides include both naturally occurring molecules and synthetic analogues. The polynucleotides described herein encompass all forms of sequences including, but not limited to, single-stranded forms, double-stranded forms, and the like.

Variants and fragments of polynucleotides that encode polypeptides that retain the desired enzymatic activity (i.e., nitrile hydratase, amidase, asparaginase, ACC deaminase, cyanoalanine synthase-like enzyme, alkane or ammonium monooxygenase, methane monooxygenase, toluene dioxygenase, or cyanidase activity) may also be used herein. By “fragment” is intended a portion of the polynucleotide and hence also encodes a portion of the corresponding protein. Polynucleotides that are fragments of an enzyme nucleotide sequence generally comprise at least 10, 15, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length enzyme polynucleotide sequence. A polynucleotide fragment will encode a polypeptide with a desired enzymatic activity and will generally encode at least 15, 25, 30, 50, 100, 150, 200, or 250 contiguous amino acids, or up to the total number of amino acids present in a full-length enzyme amino acid sequence. “Variant” is intended to mean substantially similar sequences. Generally, variants of a particular enzyme sequence will have at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the reference enzyme sequence, as determined by standard sequence alignment programs. Variant polynucleotides described herein will encode polypeptides with the desired enzyme activity. By way of example, the relatedness between two polynucleotides or two polypeptides can be described as identity. The identity between two sequences can be determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-7). The output of Needle labeled “longest identity” is used as the percent identity and is calculated as (Identical Residues (i.e., nucleotides or peptides)×100)/(Length of Alignment−Total Number of Gaps in Alignment).

As used in the context of production of transgenic cells, the term “introducing” is intended to mean presenting to a host cell, particularly a microorganism such as Escherichia coli, with a polynucleotide that encodes a nitrile hydratase, amidase, asparaginase, ACC deaminase, cyanoalanine synthase-like enzyme, alkane or ammonium monooxygenase, methane monooxygenase, toluene dioxygenase, and/or cyanidase. Optionally, the polynucleotide will be presented in such a manner that the sequence gains access to the interior of a host cell, including its potential insertion into the genome of the host cell. The disclosed methods do not depend on a particular protocol for introducing a sequence into a host cell, only that the polynucleotide gains access to the interior of at least one host cell. Methods for introducing polynucleotides into host cells are well known, including, but not limited to, stable transfection methods, transient transfection methods, and virus-mediated methods. “Stable transfection” is intended to mean that the polynucleotide construct introduced into a host cell integrates into the genome of the host and is capable of being inherited by the progeny thereof “Transient transfection” or “transient expression” is intended to mean that a polynucleotide is introduced into the host cell but does not integrate into the host's genome.

Furthermore, the nitrile hydratase, amidase, asparaginase, ACC deaminase, cyanoalanine synthase-like enzyme, alkane or ammonium monooxygenase, methane monooxygenase, toluene dioxygenase, or cyanidase nucleotide sequence may be contained in, for example, a plasmid for introduction into the host cell. Typical plasmids of interest include vectors having defined cloning sites, origins of replication, and selectable markers. The plasmid may further include transcription and translation initiation sequences and transcription and translation terminators. Plasmids can also include generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or optimally both. For general descriptions of cloning, packaging, and expression systems and methods, see Giliman and Smith, Gene 8:81-97 (1979); Roberts et al., Nature 328:731-734 (1987); Berger and Kimmel, Guide to Molecular Cloning Techniques, Methods in Enzymology, Vol. 152 (Academic Press, Inc., San Diego, California) (1989); Sambrook et al., Molecular Cloning: A Laboratory Manual, Vols. 1-3 (2d ed; Cold Spring Harbor Laboratory Press, Plainview, New York) (1989); and Ausubel et al., Current Protocols in Molecular Biology, Current Protocols (Greene Publishing Associates, Inc., and John Wiley & Sons, Inc., New York; 1994 Supplement) (1994). Transgenic host cells that express one or more of the enzymes may be used in the disclosed methods as whole cells or as a biological source from which one or more enzymes can be isolated.

Apparatuses and carriers for inhibiting or reducing fungal growth and for performing the methods disclosed are further provided. In particular embodiments, an apparatus or carrier for inhibiting or reducing fungal growth comprising a catalyst that comprises one or more bacteria selected from the group consisting of Rhodococcus spp., Pseudomonas chloroaphis, Brevibacterium ketoglutamicum, and mixtures thereof is disclosed herein. Rhodococcus rhodochrous DAP 96253 strain, Rhodococcus rhodochrous DAP 96622 strain, Rhodococcus erythropolis, or mixtures thereof may be used in certain aspects. The one or more bacteria of an apparatus or carrier are provided in a quantity sufficient to inhibit or reduce fungal growth as defined herein above. In other aspects, the catalyst comprises one or more enzymes (i.e., nitrile hydratase, amidase, asparaginase, ACC deaminase, cyanoalanine synthase-like enzyme, alkane or ammonium monooxygenase, methane monooxygenase, toluene dioxygenase, and/or cyanidase) in a quantity or at an enzymatic activity level sufficient to inhibit or reduce fungal growth. Sources of the desired enzymes for use as a catalyst in the apparatuses or carriers disclosed herein are also described in detail above. For example, the catalyst may be used in the form of whole cells that produce (or are induced or genetically modified to produce) one or more of the enzymes or may comprise the enzyme(s) themselves in an isolated, purified, or semi-purified form. A carrier for compositions for inhibiting or reducing fungal growth can, for example, be selected from the group consisting of paper, DEAE, cellulose, waxes, glutaraldehyde, and granular activated carbon. In certain aspects, other carriers such as physical structures, inanimate objects, or materials as described below may be used.

Apparatuses and carriers for inhibiting or reducing seed germination and for performing the methods disclosed are further provided. In particular embodiments, an apparatus or carrier for inhibiting or reducing seed germination comprising a catalyst that comprises one or more bacteria selected from the group consisting of Rhodococcus spp., Pseudomonas chloroaphis, Brevibacterium ketoglutamicum, and mixtures thereof is disclosed herein. Rhodococcus rhodochrous DAP 96253 strain, Rhodococcus rhodochrous DAP 96622 strain, Rhodococcus erythropolis, or mixtures thereof may be used in certain aspects. The one or more bacteria of an apparatus or carrier are provided in a quantity sufficient to inhibit or reduce seed germination as defined herein above. In other aspects, the catalyst comprises one or more enzymes (i.e., nitrile hydratase, amidase, asparaginase, ACC deaminase, cyanoalanine synthase-like enzyme, alkane or ammonium monooxygenase, methane monooxygenase, toluene dioxygenase, and/or cyanidase) in a quantity or at an enzymatic activity level sufficient to inhibit or reduce seed germination. Sources of the desired enzymes for use as a catalyst in the apparatuses or carriers disclosed herein are also described in detail above. For example, the catalyst may be used in the form of whole cells that produce (or are induced or genetically modified to produce) one or more of the enzymes or may comprise the enzyme(s) themselves in an isolated, purified, or semi-purified form. A carrier for compositions for inhibiting or reducing seed germination can, for example, be selected from the group consisting of paper, DEAE, cellulose, waxes, glutaraldehyde, and granular activated carbon. In certain aspects, other carriers such as physical structures, inanimate objects, or materials as described below may be used.

Apparatuses and carriers for inhibiting or reducing seed germination and inhibiting or reducing fungal growth and for performing the methods disclosed are further provided. In particular embodiments, an apparatus or carrier for inhibiting or reducing seed germination comprising a catalyst that comprises one or more bacteria selected from the group consisting of Rhodococcus spp., Pseudomonas chloroaphis, Brevibacterium ketoglutamicum, and mixtures thereof is disclosed herein. Rhodococcus rhodochrous DAP 96253 strain, Rhodococcus rhodochrous DAP 96622 strain, Rhodococcus erythropolis, or mixtures thereof may be used in certain aspects. The one or more bacteria of an apparatus or carrier are provided in a quantity sufficient to inhibit or reduce seed germination as defined herein above. In other aspects, the catalyst comprises one or more enzymes (i.e., nitrile hydratase, amidase, asparaginase, ACC deaminase, cyanoalanine synthase-like enzyme, alkane or ammonium monooxygenase, methane monooxygenase, toluene dioxygenase, and/or cyanidase) in a quantity or at an enzymatic activity level sufficient to inhibit or reduce seed germination. Sources of the desired enzymes for use as a catalyst in the apparatuses or carriers disclosed herein are also described in detail above. For example, the catalyst may be used in the form of whole cells that produce (or are induced or genetically modified to produce) one or more of the enzymes or may comprise the enzyme(s) themselves in an isolated, purified, or semi-purified form. A carrier for compositions for inhibiting or reducing seed germination can, for example, be selected from the group consisting of paper, DEAE, cellulose, waxes, glutaraldehyde, and granular activated carbon. In certain aspects, other carriers such as physical structures, inanimate objects, or materials as described below may be used.

Apparatuses for inhibiting seed germination encompassed by the present disclosure may be provided in a variety of suitable formats and may be appropriate for single use or multiple uses (e.g., “re-chargeable”).

In particular embodiments, the catalyst is provided in an immobilized format. Any process or matrix for immobilizing the catalyst may be used so long as the ability of the one or more bacteria (or enzymes) to inhibit seed germination is retained. For example, the catalyst may be immobilized in a matrix comprising alginate (e.g., calcium alginate), carrageenan, DEAE-cellulose, or polyacrylamide. Other such matrices are well known in the art and may be further cross-linked with any appropriate cross-linking agent, including but not limited to glutaraldehyde and/or polyethylenimine, to increase the mechanical strength of the catalyst matrix. In one aspect, the catalyst is immobilized in a glutaraldehyde cross-linked DEAE-cellulose matrix. The catalyst, particularly the catalyst in an immobilized form, may be further presented as a “catalyst module element.” A catalyst module element comprises a catalyst, such as an immobilized catalyst, within an additional structure that, for example, reduces potential contact with the catalyst, facilitates replacement of the catalyst, or permits air flow across the catalyst. Futher, matrices as described herein may be placed on, embedded in, placed in, or associated with physical structures, inanimate objects, or materials as described below.

In one embodiment, the matrix comprises alginate, or salts thereof. Alginate is a linear copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks), alternating M and G-residues (MG-blocks), or randomly organized blocks. In one embodiment, calcium alginate is used as the substrate, more particularly calcium alginate that has been cross-linked, such as with polyethylenimine, to form a hardened calcium alginate substrate. Further description of such immobilization techniques can be found in Bucke (1987) “Cell Immobilization in Calcium Alginate” in Methods in Enzymology, Vol. 135(B) (Academic Press, Inc., San Diego, Calif.; Mosbach, ed.), which is incorporated herein by reference. An exemplary method of immobilization using polyethylenimine cross-linked calcium alginate is also described below in Example 5. In another embodiment, the matrix comprises an amide-containing polymer. Any polymer comprising one or more amide groups could be used. In one embodiment, the substrate comprises a polyacrylamide polymer.

Increased mechanical strength of an immobilized catalyst matrix can be achieved through cross-linking. For example, cells can be chemically cross-linked to form agglutinations of cells. In one embodiment, cells harvested are cross-linked using glutaraldehyde. For example, cells can be suspended in a mixture of de-ionized water and glutaraldehyde followed by addition of polyethylenimine (PEI) until maximum flocculation is achieved. The cross-linked cells (typically in the form of particles formed of a number of cells) can be harvested by simple filtration. Further description of such techniques is provided in Lopez-Gallego et al. (2005) J. Biotechnol. 119:70-75, which is hereby incorporated by reference in its entirety. In certain aspects, catalytic matrices as described herein may be cross-linked with physical structures, inanimate objects, or materials as described below.

In certain aspects, the catalyst can comprise a physical structure”, “inanimate object”, or “material”. In certain aspects, the catalyst, catalyst matrix, immobilized catalyst or one or more catalyst module elements are placed in, placed on, embedded in, or affixed to a “physical structure”, “inanimate object”, or “material”. In certain aspects, the catalyst or individual components thereof may be cross-linked with a “physical structure”, “inanimate object”, or “material”. The physical form of the catalyst in these aspects can vary according to compositions as described above (for example a liquid composition, a solid composition, and the like) so long as catalytic activity to inhibit or reduce fungal growth is still maintained. The physical structure includes but is not limited to a film, sheet, coating layer, box, pouch, bag, counter top, cardboard box, an inorganic surface, paper wrapping, wallboard, wood, medical device, surgical dressing, or slotted chamber capable of holding one or more catalyst module elements. In certain embodiments, the physical structure comprises a container suitable for transport or storage of fruit, vegetables, or flowers. The physical structure may further comprise more than one individual structure, whereby all of the individual structures are connected to a central catalyst or catalyst module element. A physical structure described herein above may optionally be refrigerated by external means or comprise a refrigeration unit within the physical structure itself. By way of example, the physical structure can be a sheet or film comprising a sufficient quantity of the one or more bacteria, one or more enzymes, or enzymatic extract necessary to inhibit or reduce fungal growth. Optionally, the sheet or film is pullulan, or cellophane. Such sheets or films can be used to wrap the plant or plant part. By way of example, the film can be made of pullulan and used to wrap flowers. In certain embodiments, the physical structure comprises or is a container suitable for transport or storage of grain, e.g., a grain silo.

In certain aspects, the catalyst can comprise a physical structure”, “inanimate object”, or “material”. In certain aspects, the catalyst, catalyst matrix, immobilized catalyst or one or more catalyst module elements are placed in, placed on, embedded in, or affixed to a “physical structure”, “inanimate object”, or “material”. The physical form of the catalyst in these aspects can vary according to compositions as described above (for example a liquid composition, a solid composition, and the like) so long as catalytic activity to inhibit or reduce seed germination is still maintained. The physical structure can include but is not limited to a film, sheet, coating layer, box, pouch, bag, counter top, cardboard box, an inorganic surface, paper wrapping, wallboard, wood, medical device, surgical dressing, or slotted chamber capable of holding one or more catalyst module elements. In certain embodiments, the physical structure comprises a container suitable for transport or storage of fruit, vegetables, or flowers. The physical structure may further comprise more than one individual structure, whereby all of the individual structures are connected to a central catalyst or catalyst module element. A physical structure described herein above may optionally be refrigerated by external means or comprise a refrigeration unit within the physical structure itself. By way of example, the physical structure can be a sheet or film comprising a sufficient quantity of the one or more bacteria, one or more enzymes, or enzymatic extract necessary to inhibit or reduce seed germination. Optionally, the sheet or film is pullulan, or cellophane. Such sheets or films can be used to wrap the plant or plant part. By way of example, the film can be made of pullulan and used to wrap flowers. In certain embodiments, the physical structure comprises or is a container suitable for transport or storage of grain, e.g., a grain silo.

In certain aspects, one or more seeds or isolated seeds can be associated with, in close proximity to, or in physical contact with physical structures, inanimate objects, or materials as described above so germination thereof is reduced or inhibited.

The skilled artisan will further recognize that any of the methods, apparatuses, physical structures, compositions, or carriers disclosed herein can be combined with other known methods, apparatuses, physical structures, compositions, and carriers for inhibiting seed germination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1

Induced cells of Rhodococcus rhodochrous DAP 96253 when placed in proximity to post harvest fruit (peaches, tomatoes, grapes, strawberries) will control and significantly delay the development of mold(s) on these post-harvested fruit. In a similar manner, induced cells of Rhodococcus rhodochrous DAP 96253 inhibit the development of mold(s) on dried corn kernels under conditions which result in mold development on control corn.

As a worst-case, dried corn kernels were placed on plates containing a solidified medium (Sabouraud's Dextrose Agar [SDA]). SDA is a medium specifically formulated for the growth and propagation of molds. Corn kernels placed on SDA would be expected to promote the rapid outgrowth of mold spores resulting in visible mycelia. Under such conditions, a large percentage of the corn kernels also would be expected to germinate, further exacerbating the development of visible mold. Control kernels all showed mold growth, and most showed seed germination. Similar kernels placed on SDA but in the proximity to induced cells of R. rhodochrous DAP 96253 showed no mold growth and no seed germination.

Repetition of the above experiments all showed the same results. In subsequent experiments, the kernels exposed to the induced cells of R. rhodochrous DAP 96253, were removed from the presence of the induced cells of R. rhodochrous DAP 96253, and the kernels were subjected to conditions where germination of the kernels would be expected. These kernels did not germinate. When the kernels that had been exposed to the induced cells of R. rhodochrous DAP 96253 were briefly washed and then place under conditions which support kernel germination, the kernels germinated.

These experiments showed that the effect of the induced cells of R. rhodochrous DAP 96253 on seed germination inhibition exhibited residual activity after the R. rhodochrous cells were removed. Thus the induced R. rhodochrous DAP 96253 did not have to be always (permanently) present. Exposure for a defined period, resulted in a longer period of control. Washing of the kernels resulted in the cancellation of the seed germination inhibition.

1. A method for inhibiting seed germination, comprising exposing an isolated seed to a composition comprising one or more bacteria, wherein the one or more bacteria are selected from the group consisting of genus Rhodococcus, genus Brevibacterium, genus Pseudonocardia, genus Nocardia, genus Pseudomonas and combinations thereof, and wherein the one or more bacteria are provided in a quantity sufficient to inhibit seed germination.

2. The method of claim 1, wherein the one or more bacteria are induced to produce one or more enzymes selected from the group consisting of nitrile hydratases, amidases, asparaginases, ACC deaminases, cyanoalanine synthase-like enzymes, monooxygenases, dioxygenases, cyanidases, and combinations thereof.

3. The method of claim 1, wherein the one or more bacteria are from the genus Rhodococcus.

4. The method of claim 3, wherein the one or more bacteria are selected from the group consisting of Rhodococcus rhodochrous DAP 96253, Rhodococcus rhodochrous DAP 96622, Rhodococcus erythropolis, or combinations thereof.

5. The method of claim 2, wherein the composition further comprises the one or more enzymes or an enzymatic extract produced by the one or more bacteria.

6. The method of claim 1, wherein the composition further comprises an inducing agent selected from the group consisting of urea, methyl carbamate, methacrylamide, acetamide, cobalt, asparagine or asparagine derivative, and combinations thereof.

7. The method of claims 6, wherein the inducing agent comprises urea or methyl carbamate and one or more of cobalt and asparagine.

8. The method of claim 1, wherein the composition further comprises a stabilizing agent.

9. The method of claim 8, wherein the stabilizing agent is trehalose.

10. The method of claim 1, wherein the one or more bacteria are fixed with glutaraldehyde and cross-linked.

11. The method of claim 1, wherein the one or more bacteria are provided in a coating layer.

12. The method of claim 11, wherein the coating layer is selected from a hydrophobic fatty acid polyester coating or a wax.

13. The method of claim 1, wherein the composition comprises a magnetic material.

14. (canceled)

15. The method of claim 1, wherein the seed is indirectly exposed to the one or more bacteria.

16. The method of claim 1, wherein the seed is directly exposed to the one or more bacteria.

17-20. (canceled)

21. The method of claim 1, wherein the composition is provided in liquid form.

22. The method of claim 21, wherein the liquid is sprayed at or near the seed.

23. The method of claim 21, wherein the composition further comprises a liquid carrier.

24. The method of claim 23, wherein the liquid carrier is selected from the group consisting of an aromatic hydrocarbon, a substituted naphthalene, a phthalic acid ester, an aliphatic hydrocarbon, an alcohol, and a glycol.

25. The method of claim 1, wherein the composition is provided as a solid and the solid is applied at or near the seed.

26. The method of claim 25, wherein the solid further comprises a solid carrier.

27. The method of claim 26, wherein the solid carrier is selected from the group consisting of a dust, a wettable powder, a water dispersible granule, and a mineral filler.

28. The method of claim 27, wherein the mineral filler is selected from the group consisting of calcites, silicas, talcs, kaolins, montmorillonites, attapulgites, and mixtures thereof.

29. The method of claim 1, further comprising exposing the seed to one or more exogenous enzymes selected from the group consisting of nitrile hydratase, amidase, asparaginase, ACC deaminase, cyanoalanine synthase-like enzyme, alkane monooxygenase, ammonium monooxygenase, methane monooxygenase, toluene dioxygenase, cyanidase, and combination thereof, wherein the one or more exogenous enzymes are exposed to the plant or plant part in a quantity sufficient to inhibit seed germination.

30. (canceled)

31. A method for inhibiting seed germination, comprising exposing an isolated seed to a composition comprising one or more enzymes produced by one or more bacteria, wherein the one or more bacteria are selected from the group consisting of genus Rhodococcus, genus Brevibacterium, genus Pseudonocardia, genus Nocardia, genus Pseudomonas and combinations thereof, and wherein the one or more enzymes are provided in a quantity sufficient to inhibit seed germination.

32-58. (canceled)
